(19) United States
(12) Patent Application Publication
(10) Pub. No.: US 2015/0025534 A1
(43) Pub. Date: Jan. 22, 2015

(54) APPARATUS AND METHOD FOR BONE HARVESTING

(71) Applicant: BONE BUDDIES, LLC, Tyler, TX (US)

(72) Inventors: Charles R. Gordon, Tyler, TX (US); James E. Deaton, Georgetown, TX (US); Corey Harbold, Noonday, TX (US)

(21) Appl. No.: 14/332,974

(22) Filed: Jul. 16, 2014

Related U.S. Application Data
(60) Provisional application No. 61/847,258, filed on Jul. 17, 2013.

(51) Int. Cl. A61B 17/16

(52) U.S. Cl. CPC A61B 17/1635
USPC 606/80

(57) ABSTRACT

A bone harvesting device has a probe and a sleeve for surrounding the probe. The probe has a shaft and a handle coupled to an end of the shaft. The sleeve is a cylindrical shaft that slidably receives the probe. A second handle is coupled to the shaft. In use, the probe and sleeve are driven through tissue until the probe contacts a bone harvest site. The probe is then removed and the sleeve is pounded into the bone to harvest a bone graft. The probe may be hollow and have fenestrations so that it may also be used to harvest bone marrow.

### CROSS-REFERENCE TO RELATED APPLICATIONS

This application claims priority to U.S. Provisional Application No. 61/847,258 filed Jul. 17, 2013, the entire contents of which is specifically incorporated by reference herein without disclaimer.

### BACKGROUND

1. Field of the Invention

The present invention relates to an apparatus and method for harvesting bone marrow and bone chips.

2. Description of Related Art

Many medical procedures benefit from the use of harvested bone marrow or bone grafts. For example, spinal fusion surgery typically involves using harvested bone material to cause two vertebrae to grow together into one fused unit. The bone graft for such procedures can be obtained from a patient's own body (i.e., an autograft). Typically, the bone graft is harvested from the iliac crest of a patient's hip. The bone graft may be supplemented with bone marrow aspirate, which has the potential of obtaining fusion rates which are better than allograft alone.

There are existing devices for harvesting bone marrow and for harvesting bone grafts. However, existing devices have certain deficiencies, and there is a need for an improved device for harvesting bone marrow and bone grafts.

### SUMMARY

In accordance with an exemplary embodiment, a bone harvesting device comprises a probe and a sleeve. The probe comprises a first shaft with a distal end and a proximal end, and a first handle coupled to the proximal end of the shaft. The sleeve comprises a second shaft with a distal end and a proximal end and a second handle coupled to the proximal end of the shaft. The second shaft is configured to slidably receive the probe shaft.

In accordance with another exemplary embodiment, a bone harvesting device comprises a supporting sleeve and a probe. The supporting sleeve comprises a cylindrical sleeve with a distal end and a proximal end, and a sleeve handle coupled to the proximal end of the cylindrical sleeve. The probe comprises a cylindrical shaft with a distal end and a proximal end, and a probe handle coupled to a proximal end of the shaft. The shaft is sized to mate with the interior of the cylindrical sleeve. The probe handle has a portion for interfacing with the sleeve handle when the cylindrical shaft is inserted into the cylindrical sleeve.

In another exemplary embodiment, a method of harvesting a bone graft comprises assembling a probe and a supporting sleeve surrounding said probe, driving said probe and said sleeve into a donor site, withdrawing said probe, and utilizing said sleeve to harvest bone graft material.

The term “coupled” is defined as connected, although not necessarily directly. The terms “a” and “an” are defined as one or more unless this disclosure explicitly requires otherwise.

The terms “substantially,” “approximately,” and “about” are defined as largely but not necessarily wholly what is specified (and includes what is specified; e.g., substantially 90 degrees includes 90 degrees and substantially parallel includes parallel), as understood by a person of ordinary skill in the art. In any disclosed embodiment, the terms “substantially,” “approximately,” and “about” may be substituted with “within [a percentage] of” what is specified, where the percentage includes 0.1, 1, 5, and 10 percent.

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, a system, or a component of a system, that “comprises,” “has,” “includes” or “contains” one or more elements or features possesses those one or more elements or features, but is not limited to possessing only those elements or features. Likewise, a method that “comprises,” “has,” “includes” or “contains” one or more steps possesses those one or more steps, but is not limited to possessing only those one or more steps. Additionally, terms such as “first” and “second” are used only to differentiate structures or features, and not to limit the different structures or features to a particular order.

A device, system, or component of either that is configured in a certain way is configured in at least that way, but it can also be configured in other ways than those specifically described.

Any embodiment of any of the systems and methods can consist of or consist essentially of—rather than comprise/include/contain/have—any of the described elements, features, and/or steps. Thus, in any of the claims, the term “consisting of” or “consisting essentially of” can be substituted for any of the open-ended linking verbs recited above, in order to change the scope of a given claim from what it would otherwise be using the open-ended linking verb.

The feature or features of one embodiment may be applied to other embodiments, even though not described or illustrated, unless expressly prohibited by this disclosure or the nature of the embodiments.

Details associated with the embodiments described above and others are presented below.

### BRIEF DESCRIPTION OF THE DRAWINGS

FIG. 1 is a sectional diagram of a bone harvester in accordance with an embodiment of the present invention;

FIG. 1A is an enlarged view of the distal end of the bone harvester of FIG. 1;

FIG. 2 is a diagram of a bone marrow and bone harvester in accordance with an embodiment of the present invention; and

FIG. 2A is an enlarged view of the distal end of the bone marrow and bone harvester of FIG. 2;

FIGS. 3A, 3B and 3C are diagrams illustrating the operation of a bone harvester in accordance with an embodiment of the present invention.

### DESCRIPTION OF ILLUSTRATIVE EMBODIMENTS

In the following detailed description, reference is made to the accompanying drawings, in which are shown exemplary but non-limiting and non-exhaustive embodiments of the invention. These embodiments are described in sufficient detail to enable those having skill in the art to practice the invention, and it is understood that other embodiments may be used, and other changes may be made, without departing from the spirit or scope of the invention. The following detailed description is, therefore, not to be taken in a limiting sense, and the scope of the invention is defined only by the appended claims. In the accompanying drawings, like reference numerals refer to like parts throughout the various figures unless otherwise specified.

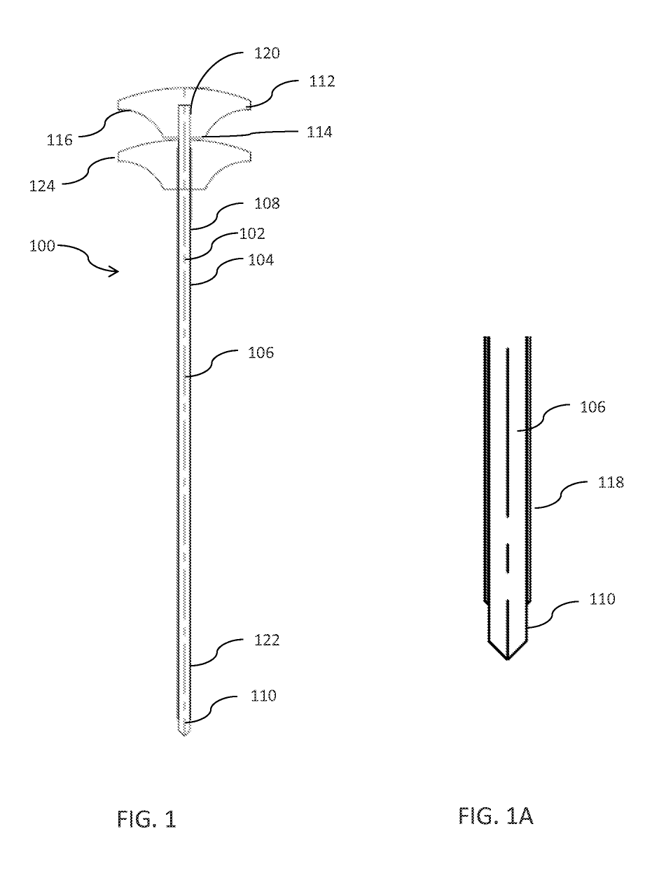

Referring to FIGS. 1 and 1A, in accordance with an illustrative embodiment, a device for harvesting bone 100 comprises a probe portion 102 and a sleeve portion 104. The probe portion 102 comprises a shaft 106 having a proximal end 108 and a distal end 110. In an exemplary embodiment, the shaft 106 is substantially solid and formed of stainless steel. In one particular embodiment, the shaft has an outer diameter of approximately 0.168-0.165 inches and is formed of 316 stainless steel. The proximal end 108 of the shaft 106 is coupled to a handle portion 112. The shaft 106 may be embedded into a hole in the handle portion 112. The handle 112 may be formed out of a medical grade material, such as a plastic material which is resistant to sterilization procedures. One suitable material is a polyphenylsulfone resin, such as the RADEL® resin available from Solvay Specialty Polymers USA, LLC, Alpharetta, Ga., USA. The handle 112 is preferably formed in a generally t-shaped configuration, with a bottom surface portion 114. The handle may be approximately 2″ in width, and may be approximately 0.62″ wide. A pair of radiused services 116 are located on the bottom portion of the handle 112 to provide ergonomic gripping areas.

The distal end 110 of the shaft 106 may be configured to promote easy delivery of the probe through tissue to the bone harvest site. In one embodiment, the distal end 110 of the shaft 106 is beveled at approximately 45 degrees. In one embodiment, the shaft 106 is approximately 8″ in length from the tip to the point where it enters the probe handle.

The sleeve portion 104 comprises a hollow shaft 118 having a proximal end 120 and a distal end 122. The shaft 118 may be formed as a hollow cylinder which is sized to receive the shaft 106 of the probe 102. In an exemplary embodiment, the shaft is formed of a 6 gauge hypotube (i.e., an outer diameter of 0.204-0.202″ and an inner diameter of 0.176-0.170″) and is formed of 304 stainless steel. The proximal end 120 of the sleeve 118 is coupled to a handle portion 124. The shaft 118 may be embedded into a hole which extends through the handle portion 124. The handle 124 may be formed of similar materials and have a similar configuration to the previously described handle 112.

The distal end 110 of the shaft 118 may be configured to promote easy delivery of the shaft 118 through tissue to the bone harvest site and subsequently through bone. In one embodiment, the distal end 110 of the shaft 118 is beveled at approximately 45 degrees. In one embodiment, the shaft 118 is approximately 7″ in length from the tip to the point where it enters the sleeve handle.

The probe 102 and sleeve 104 portions may be assembled together as shown. As seen in FIG. 1, the probe shaft 106 is long enough to extend outside the distal end 122 of the hollow shaft 118 of the sleeve portion 104. The probe shaft 106 and hollow shaft 118 sizes are chosen so that they fit together snugly. The bottom portion 114 of the handle 112 of the probe portion 1-2 mates with the top of the handle 124 of the sleeve portion 104 so that force applied to the probe handle 112 is also transmitted to the sleeve handle 124. Thus, when pressure is applied to the probe handle 112, force is transmitted to the sleeve portion 104 so that both the probe 102 and sleeve 104 move forward at the same time.

In use, the assembled device 100 is delivered to a harvest site. One suitable harvest site is the iliac crest. The operator drives the probe 102 and sleeve 104 into the bone by pressing the probe 102 forward through the soft tissue until it hits the bone. The probe 102 is then removed from the sleeve 104 by pulling on the handle. The sleeve portion 104 is then driven further into the bone to force bone into the interior of the hollow shaft 104. The operator may rotate the handle while moving it forward to aid in the harvesting process. After a sufficient amount of bone has been harvested, the sleeve, which is filled with bone graft material, is removed. The probe 102 may be reinserted into the sleeve 104 to press the harvested graft material out of the sleeve.

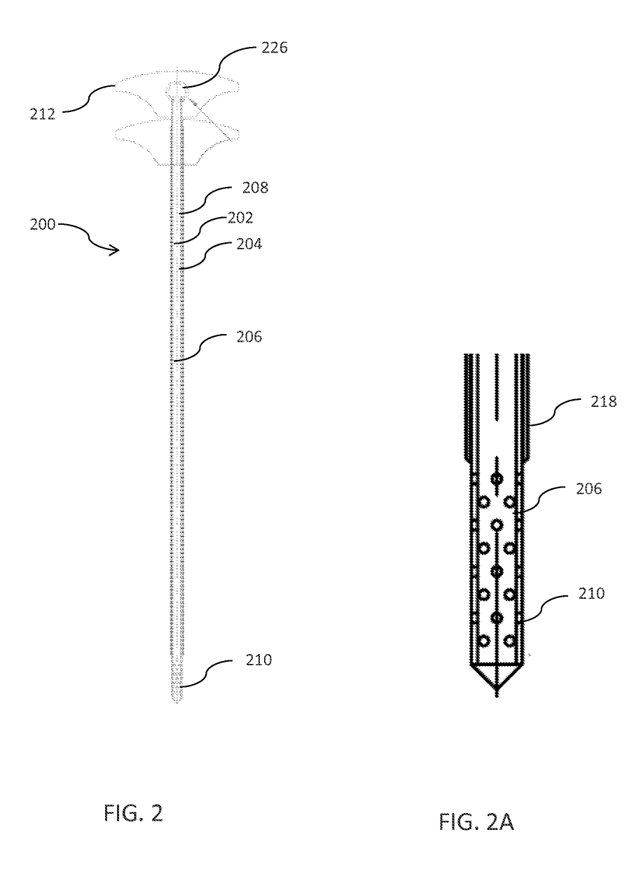

Referring to FIGS. 2 and 2A, in accordance with an illustrative embodiment, a device 200 for harvesting bone and bone marrow comprises a probe portion 202 and a sleeve portion 204. The probe portion 202 comprises a shaft 206 having a proximal end 208 and a distal end 210. In an exemplary embodiment, the shaft 206 is hollow and formed of stainless steel. In one particular embodiment, the shaft 206 has an outer diameter of approximately 0.168-0.165 inches and is formed of 316 stainless steel. The proximal end 208 of the shaft 206 is coupled to a handle portion 212. The shaft 206 may be embedded into a hole in the handle portion 212. A port 226 which is in fluid communication with the interior of the shaft 206 is disposed in the handle. In one embodiment, the port 226 comprises a luer lock for mating with a source of negative pressure, such as a syringe. The handle 224 may be formed of similar materials and have a similar configuration to the previously described handles.

The sleeve portion 204 is substantially similar to the previously described sleeve portion 104. Accordingly, a detailed description will not be repeated here for conciseness and clarity.

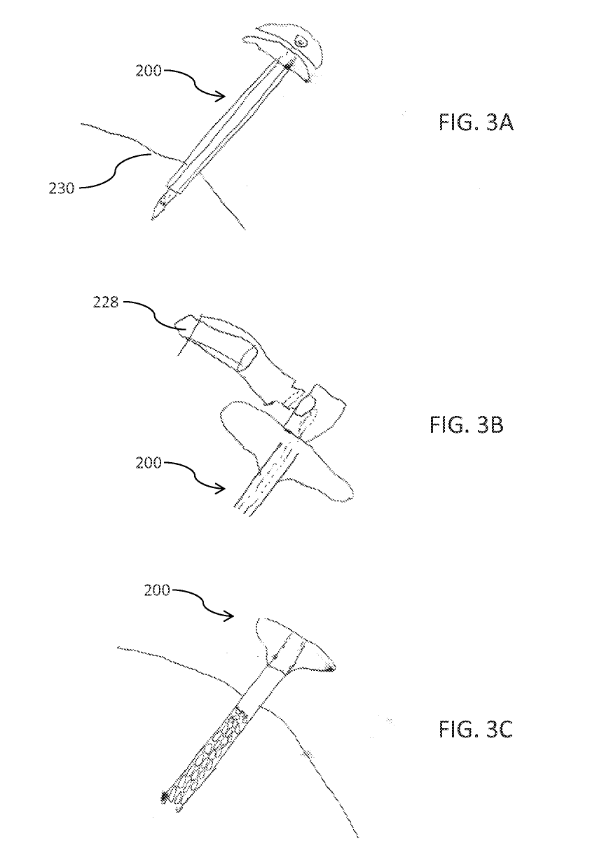

Referring to FIG. 3, in use, the assembled device 200 is delivered to a harvest site 230. One suitable harvest site is the iliac crest. The operator drives the probe 202 and sleeve 204 into the harvest site 230 by hammering on the probe handle 212 to a desired depth for harvesting marrow. A marrow collection device, such as a syringe 228, is attached to the port 226 on the probe handle 124, and marrow is harvested from the bone. The probe 202 is then removed from the sleeve 204 by pulling on the handle. The remainder of the process is substantially the same as described earlier.

The above specification and examples provide a complete description of the structure and use of exemplary embodiments. Although certain embodiments have been described above with a certain degree of particularity, or with reference to one or more individual embodiments, those skilled in the art could make numerous alterations to the disclosed embodiments without departing from the scope of this invention. As such, the various illustrative embodiments of the present devices are not intended to be limited to the particular forms disclosed. Rather, they include all modifications and alternatives falling within the scope of the claims, and embodiments other than the one shown may include some or all of the features of the depicted embodiment. For example, components may be combined as a unitary structure, and/or connections may be substituted. Further, where appropriate, aspects of any of the examples described above may be combined with aspects of any of the other examples described to form further examples having comparable or different properties and addressing the same or different problems. Similarly, it will be understood that the benefits and advantages described above may relate to one embodiment or may relate to several embodiments.

The claims are not intended to include, and should not be interpreted to include, means-plus- or step-plus-function limitations, unless such a limitation is explicitly recited in a given claim using the phrase(s) “means for” or “step for,” respectively.

What is claimed is:

1. A bone harvesting device comprising
a probe comprising:
a first shaft with a distal end and a proximal end; and
a first handle coupled to the proximal end of the shaft; and
a sleeve comprising:
a second shaft with a distal end and a proximal end, the second shaft being configured to slidably receive the probe shaft; and
a second handle coupled to the proximal end of the shaft.

2. The device of claim 1, wherein the first shaft comprises a solid rod.

3. The device of claim 1, wherein the distal end of the first shaft is beveled.

4. The device of claim 1, wherein the first and second handles are configured to interface when assembled together.

5. The device of claim 4, wherein the second handle comprises an anvil portion for interfacing with the first handle.

6. The device of claim 1, wherein the first shaft is hollow to form an interior lumen.

7. The device of claim 6, wherein the distal end of the first shaft is fenestrated.

8. The device of claim 7, further comprising a port disposed in the first handle, the port being in fluid communication with the interior lumen of the first shaft.

9. The device of claim 8, wherein the port comprises a luer lock fitting.

10. A bone harvesting device comprising
a supporting sleeve comprising:
a cylindrical sleeve with a distal end and a proximal end; and
a sleeve handle coupled to the proximal end of the cylindrical sleeve; and
a probe comprising:
a cylindrical shaft with a distal end and a proximal end, the shaft sized to mate with the interior of the cylindrical sleeve; and
a probe handle coupled to a proximal end of the shaft, the probe handle having n portion for interfacing with the sleeve handle when the cylindrical shaft is inserted into the cylindrical sleeve.

11. The bone harvesting device of claim 10, wherein the cylindrical shaft is hollow and comprises a fenestrated portion at the distal end.

12. The bone harvesting device of claim 11, wherein the probe handle comprises a luer lock fitting in fluid communication with the interior of the cylindrical shaft.

13. The bone harvesting device of claim 10, wherein the cylindrical shaft is solid.

14. A method of harvesting a bone graft, comprising:
assembling a probe and a supporting sleeve surrounding said probe;
driving said probe and said sleeve into a donor site;
withdrawing said probe; and
utilizing said sleeve to harvest bone graft material.

15. The method of claim 14, further comprising utilizing said probe to harvest bone marrow.

16. The method of claim 15, wherein said probe is hollow, and said bone marrow is harvested by applying negative pressure.

17. The method of claim 16, wherein said probe is driven to a first depth to harvest bone marrow and a second depth to harvest bone graft material.

18. The method of claim 14, wherein said donor site comprises an iliac crest.

19. The method of claim 14, further comprising driving said probe and supporting sleeve to a first depth and harvesting bone marrow.

20. The method of claim 19, further comprising applying negative pressure to said probe to harvest bone marrow.
